(19) United States
(12) Patent Application Publication
(10) Pub. No.: US 2004/0253193 A1
(43) Pub. Date: Dec. 16, 2004

(54) Oral rinse for treatment for prevention of bacterial and fungal infection

(76) Inventors: Anne Bosy, Etobicoke (CA); Julian Geller, Thornhill (CA)

Correspondence Address: EDWARDS & ANGELL, LLP, P.O. BOX 55874, BOSTON, MA, 02205, US

(21) Appl. No.: 10/487,690

(22) PCT Filed: Aug. 21, 2002
(86) PCT No.: PCT/CA02/01293

Related U.S. Application Data
(60) Provisional application No. 60/314,304, filed on Aug. 24, 2001.

(51) Int. Cl. A61K 7/22

(52) U.S. Cl. 424/54

(57) ABSTRACT

Disclosed is a mouthwash composition, and a related use and method, for the treatment or prevention of bacterial and fungal infection in the oral cavity, the mouthwash composition comprising an effective amount of metronidazole and nystatin, together with an orally-acceptable carrier.

### FIELD OF THE INVENTION

This invention relates to a mouthwash composition and method for treating or preventing bacterial and fungal infection in the oral cavity, which causes conditions such as bad breath, also referred to as halitosis or oral malodour, periodontal disease and gingival inflammation or bleeding.

### BACKGROUND OF THE INVENTION

Oral malodour (halitosis), or as it is commonly known, bad breath, is a condition that affects between 40% and 50% of the population. It is widely accepted that most individuals afflicted with oral malodour also experience psychosocial problems related to this condition. In addition to breath odour, individuals may have an unpleasant taste, described as bitter, dry, stale, fecal, metallic, hot or pasty, and they associate this taste with the presence of tainted breath, even when the mouth air has no detectable odour.

It is generally believed that the cause of this condition is due to the presence of anaerobic bacteria, especially gram-negative anaerobic bacteria, within the oral cavity. These bacteria actively degrade the sulfur-containing amino acids, methionine and cysteine, to generate pungent compounds collectively known as volatile sulfur compounds (VSC). Hydrogen sulfide (H—S—H), methyl mercaptan (CH₃—S—H) and dimethyl sulfide (CH₂—S—CH₃) are the principal odorous components generated. These substances, especially methyl mercaptan, have an unpleasant odour, even in very low concentrations, and the exhalation of these volatile sulfur compounds is perceived as bad breath.

It is also recognized that VSC can produce biologic effects such as altering the epithelial barrier within the oral cavity, resulting in bleeding and inflammation. For example, methyl mercaptan enables the penetration of bacterial toxins into the underlying connective tissue through the increased permeability of the oral mucosa. This volatile sulfur compound can alter enzymatic and immunologic activities, delay wound healing and influence gene activity through the alteration cell shape and cytoskelton pattern (Tonzetich, Bad Breath, A Multidisciplinary Approach. 79-91, 1996).

It has been established that systemic conditions, including liver and kidney abnormalities, diabetes, oral cancers, chronic sinusitis and tonsillitis, can also contribute to oral malodour. Periodontal diseases are a cluster of widespread inflammatory conditions that have an association with substantial VSC production. Oral microorganisms, predominantly gram negative anaerobic flora, contribute to the initiation and progression of periodontal diseases as well as substantial oral malodour.

Various oral rinse preparations are known for treating halitosis. U.S. Pat. No. 4,525,342 (Weiss et al.; Jun. 25, 1985) discloses a composition comprising a salt water aqueous phase and an oily phase in a double compartment double squirt bottle that allows an emulsion to be generated in the mouth during rinsing. U.S. Pat. No. 5,401,496 (Fitzig et al.; Mar. 28, 1995) discloses a preparation comprising a synthetic oil of a caprylic/capric triglyceride mixture. U.S. Pat. No. 5,738,840 (Richter; Apr. 14, 1998) discloses an aqueous composition comprising molecular chlorine dioxide and a metal chlorite salt. U.S. Pat. No. 6,071,500 (Thistle; Jun. 6, 2000) discloses a breath cleansing spray that includes xylitol as a sweetener and calcium hydroxide to raise the pH of the saliva. U.S. Pat. No. 6,132,701 (Perez et al.; Oct. 17, 2000) discloses a method for reducing halitosis that includes generating an aqueous solution of calcium hydroxide for rinsing an oral cavity. In general, known anti-halitosis mouthwashes may not remove the often bitter or pasty taste that causes distress to the individual, suggesting that they leave microorganisms in numbers large enough to produce byproducts that continue to affect the taste perceptions of the individual.

Metronidazole is used for the treatment of several types of anaerobic infections including periodontal disease. Short-term, systemic use of metronidazole administered orally in humans caused a sustained reduction of anaerobic gram-negative microorganisms, including spirochetes, Bacteroides sp., Fusobacterium sp., and the anaerobic cocci, Peptostreptococcus sp., for weeks to months, with improved periodontal health (J. Clin Periodontol. 8:29-44, 1981). Although it is well absorbed within 1 to 2 hours after ingestion, between 60 to 80% of the drug is excreted. U.S. Pat. No. 4,997,830 (van Winkelhoff et al.; Mar. 5, 1991) discloses a pharmaceutical composition comprising metronidazole and amoxicillin for the treatment of periodontitis.

However, systemic use of metronidazole can have undesirable side effects such as nausea, headaches and gastrointestinal discomfort. U.S. Pat. No. 4,568,535 (Loesche; Feb. 4, 1986) discloses a slow release film for placement in the oral cavity in a periodontal pocket, the film including metronidazole. However, such a film requires a dental professional to fit it and may be uncomfortable.

Microfungal infections of the oral cavity are a problem often associated with oral lesions and dryness. Many individuals who complain about bad breath and bad taste have substantial numbers of yeast organisms, in addition to the gram-negative anaerobic bacteria. Gingival bleeding is often also present in these individuals. The Candida species are aerobic yeasts that can also grow anaerobically. C. albicans is the species most often responsible for infections in the oral cavity and may cause a variety of disorders including gingival bleeding and denture stomatitis. Oral candidiasis is an extremely virulent and uncomfortable condition, especially prevalent in the aged and those with chronic debilitating ailments.

The establishment of a mycotic infection in the oral cavity presents a serious health problem to the individual. Thus, it is desirable to treat and contain this infection through both mechanical methods such as proper oral cleansing as well as chemical therapy in the form of antifungal drugs. However, systemic administration of antimycotics, in doses high enough to control oral infections, can induce undesirable side effects.

Nystatin is a polyene antifungal, antibiotic complex that is used for the treatment of fungal infections. Nystatin binds to the covering membrane of fungi altering the cell membrane thus leading to cell death. It is both fungicidal and fungistatic against a variety of yeasts and fungi. Nystatin is applied topically in most cases.

Canadian Patent Application 2,008,772 (Friedman; published Jul. 31, 1990) discloses a sustained-release oral antifungal varnish, that includes nystatin in a sustained-release polymer, and states that mouth rinses that include antifungal drugs do not maintain the drugs at efficacious levels in the oral cavity. U.S. Pat. No. 4,725,440 (Ridgway et al.; Feb. 16, 1988) discloses an antifungal pastille formulation for treating oral candidiasis by a relatively slow release of the antimicrobial agent, including nystatin, and indicates that use of nystatin in an oral suspension is not considered an effective way to treat oral candidiasis. PCT publication WO 99/61491 (Kolias et al.; published Dec. 2, 1999) discloses an antimicrobial denture adhesive, for the treatment of denture stomatitis, that includes nystatin as the active ingredient.

None of the above references relate to use of metronidazole and nystatin together in an oral rinse composition.

### SUMMARY OF THE INVENTION

It has not been appreciated until now that a mouthwash-based combination therapy comprising metronidazole and nystatin, as set out below, can alleviate oral conditions such as halitosis, periodontal disease and gingival inflammation or bleeding.

Therefore, the present invention provides a mouthwash composition for the treatment or prevention of bacterial and fungal infection in the oral cavity, the mouthwash composition comprising an effective amount of metronidazole and nystatin, together with an orally-acceptable carrier, such as water.

The present invention also provides a method for the treatment or prevention of bacterial and fungal infection in the oral cavity, the method comprising the step of rinsing an oral cavity with a composition comprising an effective amount of metronidazole and nystatin, together with an orally-acceptable carrier, such as water.

The present invention also provides use of an effective amount of metronidazole and nystatin for the manufacture of a product for the treatment or prevention of bacterial and fungal infection in the oral cavity.

The composition, use and/or method described herein can be used for the treatment or prevention of halitosis, a periodontal disease, or gingival inflammation or bleeding.

Metronidazole, and hence the composition, use and/or method described herein, can be used to treat bacterial infections, such as those caused by a bacterial pathogen selected from the group consisting of obligate anaerobic gram-negative bacilli such as Bacteroides sp (e.g. B. fragalis), Fusobacterium, Clostridium sp and certain anaerobic protozoal parasites such as Trichomonas, Giardia and Entamoeba. It also has bactericidal activity against the obligate anaerobic cocci isolated from infections in the oral cavity, Peptococcus sp and Peptostreptococcus sp. It is also effective in the treatment of mixed bacteria infections, that is a combination of anaerobes and aerobes.

In the oral cavity, bacterial infection is typically caused by a bacterial pathogen selected from the group consisting of Bacteroides sp, Fusobacterium sp, Spirochetes, Clostridium sp, Peptococcus sp and Peptostreptococcus sp.

Nystatin, and hence the composition, use and/or method described herein, can be used to treat fungal infections, including those caused by a Candida fungal pathogen, such as Candida albicans which can be found in oral cavity infections.

The advantages provided by the present invention include ease of use of a mouth rinse, with none of the side effects found in systemic use of both metronidazole and nystatin, and none of the inconvenience of topical formulations. Also, metronidazole and nystatin appear to act in synergy to produce positive results in patients that had not found relief from halitosis using conventional treatments. Furthermore, the composition of the present invention removes sufficient anaerobes and yeasts that an individual no longer has a perception of a bad taste.

The addition of the nystatin prevents the formation of yeast during the process of removing the anaerobes as well as eliminating yeast in those subjects where it is already present. It was observed that gingival and palatal tissue response is improved with the inclusion of nystatin in the composition, relative to metronidazole alone. Patients also reported a greater decrease in oral dryness with the inclusion of nystatin in the composition relative to metronidazole alone.

Other aspects and advantages of embodiments of the invention will be readily apparent to a person of ordinarily skilled in the art upon review of the following description.

### DETAILED DESCRIPTION OF THE PREFERRED EMBODIMENTS

The present invention relates to a vehicle that can effectively eliminate oral malodour and reduce the extent of periodontal diseases, especially the inflammatory process. The combination of metronidazole and nystatin in an aqueous suspension, when applied to the oral cavity for no less than 30 seconds several times a day, can decrease anaerobic bacteria and oral yeast within a two week period, with a noticeable difference in breath malodour and tissue status within in 24 to 48 hours.

In the composition, use and/or method of the invention, nystatin is preferably present at a concentration of about 20,000 to about 600,000 I.U. per ml (based on 100,000 I.U. per gram, this equates to a range of about 200 to about 6000 mg per ml), and is typically present at a concentration of about 100,000 I.U. per ml. Metronidazole is preferably present at a concentration of about 5 to about 200 mg per ml, more preferably between about 20 to about 50 mg per ml. One embodiment is contemplated in which, when nystatin is present at 100,000 I.U. per gram, metronidazole is not present at 50 mg per ml. Another embodiment is contemplated in which, when metronidazole is present at 50 mg per ml, nystatin is not present at 100,000 I.U. per gram.

Metronidazole (Apo-Metronidazole, Apotex Research Inc.) was prepared by grinding tablets into a powder form and water is added to make a paste. The paste was added to a suitable quantity of nystatin, which was obtained as an aqueous solution from Alimed or PMS PharmaScience (Nilstat™). Metronidazole does not dissolve in water but forms a paste that, when added to nystatin, forms a suspension. A typical 190 ml batch of oral rinse comprises 40 ml of metronidazole paste added to 150 ml nystatin solution. The mouth rinse has a slightly sticky consistency that provides effective contact with the tissues.

The composition, use and/or method of the invention can also include additives such as a corticosteroid, for example hydrocortizone at about 0.5 to about 2% per ml, which acts to promote healing of oral tissues. Pain relief agents, such as lidocaine, can also be added. In addition, a self-sterilizing agent, a flavouring agent, a colouring agent, and the like, may optionally be included as appropriate.

Preferably, a volume of about 3 to about 5 ml of the above formulation is used three times a day. If necessary, the volume and rinse frequency can be varied as appropriate. A treatment period of 14 days was found to be suitable, but this period can be varied as necessary.

Procedures

Oral hygiene procedures, including tongue cleaning, were generally performed prior to using a formulation, which was used by all adult subjects, the formulation comprising metronidazole at 50 mg per ml (25 mg per ml for children) and nystatin at 100,000 I.U. per ml.

The mouth was cleared of debris and cleansing materials by rinsing well with water. A dose of about 3 to 5 ml of the metronidazole/nystatin suspension was taken 3 times a day for a period of 14 days. The suspension was swished and gargled in the mouth for a period of 30 seconds and then expectorated. There was no rinsing or eating for at least 30 minutes after the procedure, to let the medication take affect. Normal flossing was carried out after the morning and evening rinse. With more persistent cases of interproximal bleeding or odour, after a 14 day treatment course, floss was dipped into a capful of the above-noted formulation and wet flossing carried out and the results subsequently monitored.

Clinical Results

The following tables are clinical data showing the efficacy of the nystatin-metronidazole rinse. Fifty four subjects received treatment with this medication.

Twelve of the subjects were treated with 0.2% chlorhexidine prior and evaluated for response prior to treatment with nystatin-metronidazole. These subjects used chlorhexidine twice a day rinsing for 60 seconds each time for a period of 14 days. Chlorhexidine was brushed into the tongue and carried into the gingival sulcus using an appropriate vehicle, such as floss, superfloss or proxabrush, soaked in the chlorhexidine. After the two weeks, subjects were evaluated for changes. These subjects still had some oral odours and in some cases had slightly more than the original measurements.

Subjects were instructed to refrain from food, drink and oral hygiene for two hours prior to each appointment. All subjects were instructed to refrain from garlic, onions, alcohol, spices, mouthwashes, and scented cosmetics for at least 24 hours.

The extent of the malodour for each subject has been established by taking volatile sulfur measurements in concentrations of parts per billion with a Halimeter™, a portable sulfur monitor adapted for oral use by Interscan Corporation, Chatsworth, Calif. It is relatively accurate instrument for the measurement of hydrogen sulfide but measures methyl mercaptan to about a 50% accuracy and dimethyl sulfide to an even lesser degree. When the predominant odour is methyl mercaptan, there is usually a low reading on the Halimeter when compared to the organoleptic rating.

Organoleptic ratings of mouth air, nasal air, tongue base, tongue dorsum and floss were established by three trained, experienced judges. Two criteria, bleeding on probing and measurement of periodontal pockets, were used to determine the extent of gingival bleeding and periodontal status. A calibrated probe was used to assess the probing depth of a sulcus or periodontal pocket. The rod-shaped working end of the probe is marked in millimeter units and is similar to a ruler. The probe was inserted into the sulcus or pocket and the depth measured from the junction of the tooth and gum tissue (epithelial attachment) to the top of the gum or gingival margin. Healthy gums or gingiva have pockets that range from 0.5 to 3.0 mm in depth. A depth greater than 3.0 mm is called a periodontal pocket. To measure the depth of the sulcus or pocket, the probe was held lightly and the working end of the instrument placed against the enamel and gently inserted beneath the gum until there was a soft but resilient resistance. The probe was walked around the entire circumference of the tooth and measurements of 4 mm and greater were recorded. There are six areas at which measurements were taken, 3 on the surface next to the cheek and 3 on the surface next to the tongue. These evaluations were performed just before treatment and fourteen days after treatment for all subjects except where stated otherwise.

Some subjects were evaluated 24 to 48 hours after taking the nystatin-metronidazole rinse because they travelled a significant distance (e.g. South America) and could not stay for the 14 day term. However, they were required to complete the nystatin-metronidazole rinse and report by telephone or email as to further progress.

Tables 1A, 1B and 1C show the pretreatment and post-treatment levels of odours, periodontal involvement and microbiology of subjects who were treated with a commercially available aqueous 0.2% chlorhexidine solution (PerioWorks, Vernon, B C). Table 1D shows the measurements of these same subjects following treatment with the nystatin-metronidazole formulation detailed above.

Tables 2A, 2B and 2C show the pretreatment and post-treatment levels of odours, periodontal involvement and microbiology of subjects immediately following measurements on the first appointment, and then following two weeks of treatment with the nystatin-metronidazole formulation detailed above.

Abbreviations Used in Tables

Org.m—organoleptic assessment of mouth air.

Org.tb—organoleptic assessment of the base of the tongue.

Org.td—organoleptic assessment of tongue dorsum.

Org.f—organoleptic assessment of floss odours.

nr—not recorded.

spiro—Spirochetes

fuso—Fusobacterium

gr—bacilli—Gram negative bacilli

gr—cocci—Gram negative cocci

gr—coccobacilli—Gram negative coccobacilli

Organoleptic measurements use the scale 0-5, with the following meanings: 0— no odour; 1—slightly perceivable and fleeting odour; 2—mild but definite; 3—moderate; 4—strong; 5—very strong. The pocket depth (in mm) is stated, followed by the number of pockets in brackets.

TABLE 1A
VSC and organoleptic assessments of individuals with
oral malodour prior to treatment with chlorhexidine.

| Patient | Sex | Age | VSC | Org.m | Org.tb | Org.td | Org.f | Taste | Bleeding | Pockets (mm) |
|---|---|---|---|---|---|---|---|---|---|---|
| AY2596 | F | 29 | 53 | 2 | 3 | 1 | 5 | bitter | no | no |
| SB2400 | M | 29 | 62 | 3 | 3 | 0 | 2 | dry | no | no |
| HE2046 | M | 57 | 51 | 4 | 5 | 4 | 5 | none | no | no |
| KT2016 | F | 25 | 82 | 4 | 4 | 4 | 2 | bad | no | 6(3) |
| ZT2557 | M | 40 | 36 | 1 | 0 | 1 | 0 | none | no | 4(1) |
| JM2142 | F | 35 | 47 | 2 | 1 | 3 | 0 | bitter | no | no |
| KN2036 | M | 39 | 41 | 4 | nr | 2 | 5 | dry | no | no |
| CD2416 | F | 33 | 79 | 3 | 4 | 2 | 0 | sour | no | no |
| EC2010 | F | 54 | 93 | 4 | 5 | 3 | 5 | bad | no | 4(1) |
| VC2399 | M | 37 | 134 | 3 | 3 | 2 | 2 | none | yes | no |
| JA2021 | F | 47 | 45 | 3 | 4 | 3 | 2 | none | no | 4(1) |
| TB2425 | M | 18 | 39 | 3 | 2 | 0 | 0 | manure | no | no |

TABLE 1B
Microbiology of the tongue and interproximal plaque of individuals
in Table 1A, assessed by a commercial laboratory prior to treatment
with 0.2% chlorhexidine.

| Patient | Sex | Age | spiro | fuso | gr-bacilli | gr-cocci | gr-coccobacilli | yeast |
|---|---|---|---|---|---|---|---|---|
| AY2596 | F | 29 | yes | no | yes | yes | yes | no |
| SB2400 | M | 29 | no | no | yes | yes | no | no |
| HE2046 | M | 57 | yes | yes | yes | yes | no | no |
| KT2016 | F | 25 | yes | no | yes | yes | no | yes |
| ZT2557 | M | 36 | yes | no | yes | no | no | yes |
| JM2142 | F | 35 | yes | yes | yes | yes | no | yes |
| KN2036 | M | 39 | yes | yes | yes | yes | no | yes |
| CD2416 | F | 33 | yes | no | yes | yes | no | no |
| EC2010 | F | 54 | yes | yes | yes | yes | no | yes |
| VC2399 | M | 37 | yes | no | yes | yes | yes | no |
| JA2021 | F | 47 | yes | yes | yes | yes | no | yes |
| TB2425 | M | 18 | no | no | yes | no | no | no |

TABLE 1C
VSC and organoleptic assessments of individuals from Table 1A
post treatment with a 0.2% chlorhexidine rinse for two weeks.

| Patient | Sex | Age | VSC | Org.m | Org.tb | Org.td | Org.f | Taste | Bleeding | Pockets (mm) |
|---|---|---|---|---|---|---|---|---|---|---|
| AY2596 | F | 29 | 42 | 2 | nr | 2 | 0 | bitter | no | no |
| SB2400 | M | 29 | 53 | 0 | 3 | 0 | 0 | none | no | no |
| HE2046 | M | 57 | 26 | 4 | nr | 4 | 4 | none | no | no |
| KT2016 | F | 25 | 37 | 0 | 0 | 2 | 2 | nr | no | nr |
| JM2142 | F | 35 | 116 | 0 | nr | 2 | 0 | nr | no | no |
| KN2036 | M | 39 | 43 | 0 | nr | 0 | 5 | none | no | no |
| CD2416 | F | 33 | 112 | 3 | 3 | 2 | 0 | nr | no | no |
| EC2010 | F | 54 | 149 | 3 | nr | 3 | 3 | improved | no | nr |
| VC2399 | M | 37 | 50 | 2 | 3 | 2 | 0 | none | no | no |
| JA2021 | F | 47 | 31 | 3 | nr | 1 | 3 | none | no | nr |
| TB2425 | M | 18 | 89 | 3 | nr | 3 | 2 | dry | no | no |

TABLE 1D
VSC and organoleptic assessments of individuals from
Table 1A post treatment with the nystatin-metronidazole
rinse following the two week 0.2% chlorhexidine rinsing.

| Patient | Sex | Age | VSC | Org.m | Org.tb | Org td | Org.f | Taste | Bleeding | Pockets (mm) |
|---|---|---|---|---|---|---|---|---|---|---|
| AY2596 | F | 29 | 52 | 0 | nr | 0 | 0 | none | no | no |
| SB2400 | M | 29 | 31 | 0 | 0 | 0 | 0 | none | no | no |
| HE2046 | M | 57 | 34 | 0 | nr | 0 | 0 | none | no | no |
| KT2016 | F | 25 | 30 | 0 | 0 | 0 | 0 | none | no | nr |
| ZT2557 | M | 36 | 30 | 0 | 0 | 0 | 0 | none | no | nr |
| JM2142 | F | 35 | 55 | 0 | 0 | 0 | 0 | nr | no | no |
| KN2036 | M | 39 | 41 | 0 | nr | 0 | 0 | none | no | no |
| CD2416 | F | 33 | 39 | 2¹ | nr | 0 | 0 | none | no | no |
| EC2010 | F | 54 | 39 | 0 | nr | 0 | 0 | none | no | nr |
| VC2399 | M | 37 | 36 | 2¹ | nr | 0 | 0 | none | no | no |
| JA2021 | F | 47 | 27 | 0 | 0 | 0 | 0 | none | no | nr |
| TB2425 | M | 18 | 44 | 0 | 0 | 0 | 0 | none | no | no |

¹Garlic odour from diet.

TABLE 2A
VSC and organoleptic assessments of individuals with oral malodour,
prior to treatment with the nystatin-metronidazole formulation.

| Patient | Sex | Age | VSC | Org.m | Org.tb | Orgtd | Org.f | Taste | Bleeding | Pockets (mm) |
|---|---|---|---|---|---|---|---|---|---|---|
| CW2714 | F | 24 | 362 | 5 | 5 | 5 | 4 | dry | no | no |
| FA2221 | M | 45 | 127 | 5 | 4 | 4 | 3 | metallic | no | 4 |
| RN1464 | F | 52 | 59 | 5 | 4 | 4 | 0 | nr | no | no |
| DM2413 | M | 44 | 61 | 3 | 3 | 3 | 0 | bitter | no | 4 (1) |
| MG2105 | F | 58 | 67 | 5 | 2 | 5 | 5 | sour | no | no |
| GK2809 | F | 53 | 78 | 2 | 2 | 2 | 3 | thick | yes | 5 (6) |
| RL2646 | M | 35 | 212 | 4 | 4 | 4 | 2 | metallic | no | no |
| SL2510 | F | 52 | 49 | 4 | 5 | 4 | 3 | none | nr | nr |
| JP2600 | M | 24 | 64 | 5 | 5 | 5 | 2 | acrid | yes | no |
| EP2799 | M | 50 | 198 | 3 | 4 | 3 | 2 | none | no | no |
| AR2620 | F | 34 | 115 | 3 | 4 | 1 | 3 | metallic | yes | 4 (many) |
| GW2158 | F | 36 | 81 | 4 | 3 | 4 | 3 | strong | no | no |
| GB2808 | F | 21 | 283 | 4 | 4 | 2 | 0 | sour | yes | 4 (6) |
| MC2648 | M | 35 | 65 | 5 | 5 | 3 | 2 | metallic | no | 5 (2) |
| LP2546 | F | 52 | 84 | 3 | 3 | 1 | 1 | pasty | no | 4 (2) |
| GK2712 | F | 68 | 394 | 4 | 5 | 5 | 5 | bitter | yes | no |
| SB2679 | F | 33 | 70 | 4 | 4 | 1 | 4 | foul | no | no |
| CG2304 | F | 54 | 135 | 3 | 4 | 3 | 5 | foul | no | no |
| RJ2386 | M | 39 | 55 | 1 | 4 | 0 | 2 | none | yes | 4 (2) |
| CB2564 | F | 36 | 308 | 5 | 5 | 2 | 2 | bitter | no | no |
| MH2009 | M | 8 | 55 | 3 | 3 | 2 | 0 | none | no | no |
| NG2411 | M | 50 | 142 | 4 | 5 | 3 | 2 | acid | no | 5 (3), 7 (1) |
| HT2384 | M | 81 | 38 | 3 | 4 | 1 | 5 | bad | yes | 4 (3), 5 (3) |
| PB2914 | M | 36 | 60 | 1 | 3 | 1 | 1 | dry | yes | 4 (4) |
| RK2950 | F | 51 | 56 | 2 | 3 | 1 | 2 | stale | yes | 4 (2), 5 (4) |
| BB2747 | F | 43 | 62 | 4 | nr | 0 | 5 | burning | yes | 5 (4), 4 (2) |
| FB2429 | F | 41 | 144 | 4 | 4 | 3 | 5 | stale | yes | no |
| MA2826 | F | 55 | 30 | 0 | 3 | 0 | 3 | metallic | yes | 5 (2), 4 (1) |
| PE2195 | M | 62 | 129 | 4 | 3 | 3 | 0 | none | no | 5 (1) |
| SDR2579 | M | 45 | 568 | 5 | 5 | 3 | 4 | acid | no | no |
| EE2314 | F | 50 | 210 | 5 | 4 | 4 | 5 | metallic | yes | many |
| CF2689 | M | 39 | 900 | 5 | 5 | 5 | 3 | none | yes | many |
| MS2784 | F | 27 | 208 | 4 | 5 | 4 | 0 | bitter | yes | 4 (2) |
| PS2762 | M | 31 | 101 | 4 | 4 | 3 | 2 | none | yes | 4 (6), 5 (4) |
| DL2390 | F | 25 | 66 | 0 | 4 | 3 | 2 | metallic | no | 4 (8) |
| SN2835 | F | 27 | 08 | 4 | 5 | 3 | 2 | metallic | yes | no |
| LM2862 | F | 26 | 139 | 3 | 4 | 3 | 0 | bitter | yes | no |
| RM2452 | M | 44 | 85 | 4 | 2 | 3 | 1 | metallic | yes | 5 (2) |
| MF2665 | M | 44 | 75 | 4 | 4 | 2 | 3 | metallic | no | no |
| BF2812 | M | 32 | 145 | 3 | 4 | 3 | 0 | sulfuric | yes | 5 (4), 4 (2) |
| JF2887 | M | 41 | 243 | 4 | 3 | 3 | 0 | none | no | 4 (3) |
| MG2890 | M | 38 | 260 | 4 | 4 | 3 | 3 | tinny | yes | no |
| EG2701 | M | 26 | 220 | 3 | 4 | 2 | 2 | sulfuric | no | no |
| LG2367 | F | 26 | 86 | 4 | 4 | 4 | 3 | stale | yes | 5 (2) |
| AG2558 | F | 46 | 66 | 3 | 3 | 1 | 5 | none | yes | no |
| MG2793 | F | 34 | 152 | 3 | 4 | 3 | 3 | bitter | yes | no |
| MG2709 | F | 25 | 198 | 5 | 4 | 5 | 5 | stale | yes | 4 (1), 5 (5) |
| AG2338 | M | 40 | 223 | 5 | 5 | 4 | 5 | bitter | no | 4 (2), 5 (2) |
| MH2361 | M | 43 | 82 | 5 | 5 | 5 | 3 | stale | no | 4 (3) |
| SM2954 | M | 27 | 110 | 3 | 3 | 3 | 3 | dry | yes | no |
| GL2952 | F | 3781 | 3 | 3 | 3 | 3 | 2 | bitter | no | no |

TABLE 2B
VSC and organoleptic assessments of individuals with oral malodour
prior to treatment with the nystatin-metronidazole formulation.

| Patient | Sex | Age | Spiro | fuso | gr- bacilli | gr- cocci | gr- coccobacilli | yeast |
|---|---|---|---|---|---|---|---|---|
| CW2714 | F | 24 | no | no | yes | no | no | no |
| FA2221 | M | 45 | yes | yes | yes | yes | yes | yes |
| RN1464 | F | 52 | yes | yes | yes | yes | no | yes |
| DM2413 | M | 44 | no | no | yes | yes | no | no |
| MG2105 | F | 58 | yes | yes | yes | yes | no | yes |
| GK2809 | F | 53 | yes | yes | yes | no | no | no |
| RL2646 | M | 35 | yes | yes | yes | yes | no | yes |
| SL2510 | F | 52 | yes | no | yes | yes | no | yes |
| JP2600 | M | 24 | no | no | yes | yes | no | yes |
| EP2799 | M | 50 | yes | yes | yes | no | no | no |
| AR2620 | F | 34 | yes | no | yes | yes | no | no |
| GW2158 | F | 36 | yes | yes | yes | yes | no | yes |
| GB2808 | F | 21 | no | yes | yes | no | yes | no |
| MC2648 | M | 35 | yes | no | yes | yes | no | yes |
| LP2546 | F | 52 | no | no | yes | yes | no | yes |
| GK2712 | F | 68 | yes | no | yes | yes | yes | no |
| SB2679 | F | 33 | yes | no | yes | yes | no | no |
| CG2304 | F | 54 | yes | no | yes | yes | no | no |
| RJ2386 | M | 39 | yes | no | yes | yes | no | no |
| CB2564 | F | 36 | no | no | yes | yes | no | no |
| MH2009 | M | 8 | no | no | yes | no | yes | no |
| NG2411 | M | 50 | yes | no | yes | yes | no | no |
| HT2384 | M | 81 | yes | no | yes | yes | no | no |
| PB2914 | M | 36 | no | yes | yes | no | yes | yes |
| RK2950 | F | 51 | no | yes | yes | no | yes | no |
| BB2747 | F | 43 | yes | no | yes | yes | no | no |
| FB2429 | F | 41 | yes | no | yes | yes | no | no |
| MA2826 | F | 55 | yes | yes | yes | no | no | no |
| PE2195 | M | 62 | yes | yes | yes | yes | no | yes |
| SDR2579 | M | 45 | yes | no | yes | yes | no | yes |
| EE2314 | F | 50 | yes | yes | yes | yes | yes | yes |
| CF2689 | M | 39 | yes | no | yes | yes | no | no |
| MS2784 | F | 27 | no | yes | yes | no | no | no |
| PS2762 | M | 31 | no | no | yes | yes | no | no |
| DL2390 | F | 25 | no | no | yes | yes | yes | no |
| SN2835 | F | 27 | yes | yes | yes | no | no | no |
| LM2862 | F | 26 | yes | yes | yes | no | no | no |
| RM2452 | M | 44 | yes | no | yes | no | yes | no |
| MF2665 | M | 44 | yes | no | yes | yes | no | yes |
| BF2812 | M | 32 | no | yes | yes | no | no | no |
| JF2887 | M | 41 | yes | yes | yes | no | no | no |
| MG2890 | M | 38 | no | yes | yes | no | no | no |
| EG2701 | M | 26 | yes | no | yes | no | no | no |
| LG2367 | F | 26 | yes | yes | yes | yes | no | no |
| AG2558 | F | 46 | no | no | yes | yes | no | yes |
| MG2793 | F | 34 | yes | yes | yes | no | no | yes |
| MG2709 | F | 25 | yes | no | yes | yes | no | no |
| AG2338 | M | 40 | yes | no | yes | no | no | no |
| MH2361 | M | 43 | yes | no | yes | no | no | no |
| SM2954 | M | 27 | yes | yes | yes | no | no | no |
| GL2952 | F | 37 | no | yes | yes | no | yes | no |

TABLE 2C
VSC and organoleptic assessments of individuals from Table 2A
post treatment with the nystatin-metronidazole formulation.

| Patient | Sex | Age | VSC | Org.m | Org.tb | Org td | Org.f | Taste | Bleeding | Pockets (mm) |
|---|---|---|---|---|---|---|---|---|---|---|
| CW2714 | F | 24 | 28 | 0 | 0 | 0 | 0 | none | no | no |
| FA2221 | M | 45 | 37 | 0 | 0 | 0 | 0 | none | no | no |
| RN1464 | F | 52 | 40 | 0 | 0 | 0 | 0 | none | no | no |
| DM2413 | M | 44 | 30 | 0 | nr | 0 | 0 | none | no | no |
| MG2105 | F | 58 | 34 | 0 | nr | 0 | 0 | none | no | no |
| GK2809 | F | 53 | 19 | 0 | 0 | 0 | 0 | none | improved | nr |
| RL2646 | M | 35 | 50 | 0 | 0 | 1 | 0 | none | no | no |
| SL2510 | F | 52 | 13 | 0 | 0 | 0 | 4 | none | improved | nr |
| JP2600 | M | 24 | 43 | 0 | 0 | 0 | 0 | none | no | no |
| EP2799 | M | 50 | 28 | 0 | 3 | 0 | 0 | none | no | no |
| AR2620 | F | 34 | 38 | 0 | 0 | 0 | 0 | none | improved | improved |
| GW2158 | F | 36 | 42 | 0 | 0 | 0 | 0 | none | no | no |
| GB2808 | F | 21 | 22 | 0 | 1 | 0 | 1 | none | improved | improved |
| MC2648 | M | 35 | 55 | 0 | 0 | 0 | 0 | none | no | nr |
| LP2546 | F | 52 | 42 | 0 | 0 | 0 | 0 | none | no | nr |
| GK2712 | F | 68 | 20 | 0 | 1 | 0 | 3 | none | improved | no |
| SB2679 | F | 33 | 45 | 0 | 0 | 0 | 2 | none | no | no |
| CG2304 | F | 54 | 12 | 0 | 0 | 0 | 0 | none | no | no |
| RJ2386 | M | 39 | 34 | 0 | 0 | 0 | 0 | yes | improved | improved |
| CB2564 | F | 36 | 35 | 0 | 0 | 0 | 0 | none | no | no |
| MH2009 | M | 8 | 25 | 1 | 1 | 0 | 0 | none | no | no |
| NG2411¹ | M | 50 | 41 | 0 | 0 | 0 | 4 | none | improved | nr |
| HT2384 | M | 81 | 38 | 0 | 0 | 0 | 4 | none | improved | improved |
| PB2914 | M | 36 | 50 | 0 | 1 | 1 | 0 | dry | no | 4 (2) |
| RK2950 | F | 51 | 10 | 0 | 0 | 0 | 0 | none | improved | nr |
| BB2747 | F | 43 | 24 | 0 | 0 | 0 | 0 | none | improved | nr |
| FB2429 | F | 41 | 28 | 0 | nr | 0 | 2 | none | improved | no |
| MA2826 | F | 55 | 26 | 0 | nr | 0 | 0 | none | improved | nr |
| PE2195 | M | 62 | 32 | 0 | nr | 0 | 0 | none | no | improved |
| SDR2579 | M | 45 | 41 | 0 | 0 | 0 | 0 | none | no | no |
| EE2314 | F | 50 | 19 | 0 | 0 | 0 | 2 | none | no | improved |
| CF2689 | M | 39 | 14 | 0 | nr | 0 | 0 | none | improved | nr |
| MS2784 | F | 27 | 21 | 0 | nr | 0 | 0 | none | improved | nr |
| PS2762 | M | 31 | 21 | 0 | nr | 0 | 0 | none | no | nr |
| DL2390 | F | 25 | 41 | 0 | 0 | 0 | 0 | none | no | improved |
| SN2835 | F | 27 | 30 | 0 | 0 | 0 | 0 | none | improved | no |
| LM2862 | F | 26 | 16 | 0 | 1 | 0 | 0 | none | improved | no |
| RM2452 | M | 44 | 37 | 1 | 1 | 0 | 0 | none | improved | nr |
| MF2665 | M | 44 | 45 | 1 | 1 | 0 | 0 | none | no | no |
| BF2812 | M | 32 | 28 | 0 | nr | 0 | 2 | none | improved | nr |
| JF2887 | M | 41 | 16 | 0 | 1 | 0 | 0 | none | no | improved |
| MG2890 | M | 38 | 36 | 0 | 0 | 0 | 1 | none | no | no |
| EG2701 | M | 26 | 34 | 0 | 0 | 0 | 0 | none | no | no |
| LG2367 | F | 26 | 42 | 1 | 1 | 0 | 0 | none | no | nr |
| AG2558 | F | 46 | 46 | 0 | 1 | 0 | 0 | none | no | no |
| MG2793 | F | 34 | 32 | 0 | 0 | 0 | 0 | none | improved | no |
| MG2709 | F | 25 | 20 | 0 | 1 | 0 | 5 | none | improved | improved |
| AG2338 | M | 40 | 28 | 0 | 0 | 0 | 0 | none | no | nr |
| MH2361 | M | 43 | 28 | 0 | 0 | 0 | 0 | none | no | improved |
| SM2954 | M | 27 | 21 | 0 | 0 | 0 | 0 | none | no | no |
| GL2952 | F | 37 | 14 | 1 | 0 | 0 | 0 | none | no | no |

¹Measurement were taken 24 hours after medication was first taken.

Many modifications and variations are possible and would be apparent to a person skilled in the art in light of the above teachings. It is therefore to be understood that the invention can be practiced otherwise than as specifically described herein and still will be within the spirit and scope of the appended claims.

1. A mouthwash composition formulated for the treatment or prevention of bacterial and fungal infection in the oral cavity, the mouthwash composition comprising an effective amount of metronidazole and nystatin, together with an orally-acceptable carrier.

2. A mouthwash composition formulated for the treatment or prevention of bacterial and fungal infection in the oral cavity, the mouthwash composition comprising an effective amount of metronidazole and nystatin, together with an orally-acceptable carrier, with the provision that metronidazole is not present at a concentration of 50 mg/ml when nystatin is present at a concentration of 100,000 I.U./ml and nystatin is not present at a concentration of 100,000 I.U./ml when metronidazole is present at a concentration of 50 mg/ml.

3. A mouthwash composition according to claim 1 or 2, wherein metronidazole is present at a concentration of about 5 to about 200 mg per ml and wherein nystatin is present at a concentration of about 20,000 to about 600,000 I.U. per ml.

4. A mouthwash composition according to claim 1 or 2, wherein the composition is an aqueous suspension.

5. A mouthwash composition according to claim 3, wherein the composition is an aqueous suspension.

6. A mouthwash composition according to claim 1 or 2, formulated for the treatment or prevention of halitosis.

7. A mouthwash composition according to claim 3, formulated for the treatment or prevention of halitosis.

8. A mouthwash composition according to claim 1 or 2, formulated for the treatment or prevention of a periodontal disease.

9. A mouthwash composition according to claim 3, formulated for the treatment or prevention of a periodontal disease.

10. A mouthwash composition according to claim 1 or 2, formulated for the treatment or prevention of gingival inflammation or bleeding.

11. A mouthwash composition according to claim 3, formulated for the treatment or prevention of gingival inflammation or bleeding.

12. A mouthwash composition according to claim 1 or 2, wherein the bacterial infection is caused by a bacterial pathogen selected from the group consisting of Bacteroides sp, Fusobacterium sp, Spirochetes, Clostridium sp, Peptococcus sp and Peptostreptococcus sp.

13. A mouthwash composition according to claim 3, wherein the bacterial infection is caused by a bacterial pathogen selected from the group consisting of Bacteroides sp, Fusobacterium sp, Spirochetes, Clostridium sp, Peptococcus sp and Peptostreptococcus sp.

14. A mouthwash composition according to claim 1 or 2, wherein the fungal infection is caused by a Candida fungal pathogen.

15. A mouthwash composition according to claim 3, wherein the fungal infection is caused by a Candida fungal pathogen.

16. A composition according to claim 1 or 2, further comprising a corticosteroid.

17. A composition according to claim 3, further comprising a corticosteroid.

18. A composition according to claim 16, wherein the corticosteroid is hydrocortisone.

19. A composition according to claim 17, wherein the corticosteroid is hydrocortisone.

20. A composition according to claim 1 or 2, further comprising a pain relief agent.

21. A composition according to claim 3, further comprising a pain relief agent.

22. A composition according to claim 16, further comprising a pain relief agent.

23. A composition according to claim 17, further comprising a pain relief agent.

24. A composition according to claim 20, wherein said pain relief agent is lidocaine.

25. A composition according to claim 21, wherein said pain relief agent is lidocaine.

26. A composition according to claim 22, wherein said pain relief agent is lidocaine.

27. A composition according to claim 23, wherein said pain relief agent is lidocaine.

28. A method for the treatment or prevention of bacterial and fungal infection in the oral cavity, the method comprising the step of rinsing an oral cavity with a composition comprising an effective amount of metronidazole and nystatin, together with an orally-acceptable carrier.

29. A method for the treatment or prevention of bacterial and fungal infection in the oral cavity, the method comprising the step of rinsing an oral cavity with a composition comprising an effective amount of metronidazole and nystatin, together with an orally-acceptable carrier, with the provision that metronidazole is not present at a concentration of 50 mg/ml when nystatin is present at a concentration of 100,000 I.U./ml and nystatin is not present at a concentration of 100,000 I.U./ml when metronidazole is present at a concentration of 50 mg/ml.

30. The method of claim 28 or 29, wherein metronidazole is present at a concentration of about 5 to about 200 mg per ml and wherein nystatin is present at a concentration of about 20,000 to about 600,000 I.U. per ml.

31. A method according to claim 28 or 29, wherein the composition is applied to treat or prevent halitosis.

32. A method according to claim 28 or 29, wherein the composition is applied to treat or prevent a periodontal disease.

33. A method according to claim 28 or 29, wherein the composition is applied to treat or prevent gingival inflammation or bleeding.

34. A method according to claim 28 or 29, wherein the bacterial infection is caused by a bacterial pathogen selected from the group consisting of Bacteroides sp, Fusobacterium sp, Clostridium sp, Spirochetes, Peptococcus sp and Peptostreptococcus sp.

35. A method according to claim 28 or 29, wherein the fungal infection is caused by a Candida fungal pathogen.

36-38. (Cancelled).

39. A method according to claim 28 or 29, wherein the composition is an aqueous suspension.

40-44. (Cancelled).

45. A method according to claim 28 or 29, wherein the composition further comprises a corticosteroid.

46. A method according to claim 45, wherein the corticosteroid is hydrocortisone.

47. A method according to claim 28 or 29, wherein the composition further comprises a pain relief agent.

48. A method according to claim 47, wherein said pain relief agent is lidocaine.

49. A method according to claim 30, wherein the composition is applied to treat or prevent halitosis.

50. A method according to claim 30, wherein the composition is applied to treat or prevent a periodontal disease.

51. A method according to claim 30, wherein the composition is applied to treat or prevent gingival inflammation or bleeding.

52. A method according to claim 30, wherein the bacterial infection is caused by a bacterial pathogen selected from the group consisting of Bacteroides sp, Fusobacterium sp, Clostridium sp, Spirochetes, Peptococcus sp and Peptostreptococcus sp.

53. A method according to claim 30, wherein the fungal infection is caused by a Candida fungal pathogen.

54. A method according to claim 30, wherein the composition is an aqueous suspension.

55. A method according to claim 30, wherein the composition further comprises a corticosteroid.

56. A method according to claim 55, wherein the corticosteroid is hydrocortisone.

57. A method according to claim 30, wherein the composition further comprises a pain relief agent.

58. A method according to claim 57, wherein said pain relief agent is lidocaine.
